SEQUENCE LISTING (4 sequences)

SEQ ID NO: 1 - protein, 9 aa, Artificial Sequence
  Synthetic
YLSGANLNL

SEQ ID NO: 2 - protein, 10 aa, Artificial Sequence
  Synthetic
VLSNDVCAQV

SEQ ID NO: 3 - protein, 10 aa, Artificial Sequence
  Synthetic
ALWGQDVTSV

SEQ ID NO: 4 - protein, 9 aa, Artificial Sequence
  Synthetic
WLLPGTSTL

(19) United States
(12) Patent Application Publication
(10) Pub. No.: US 2019/0183870 A1
(43) Pub. Date: Jun. 20, 2019

(54) COMBINATION OF HISTONE DEACETYLASE INHIBITOR AND IMMUNOTHERAPY

(71) Applicant: The United States of America, as represented by Secretary, Dept. of Health and Human Services, Bethesda, MD (US)

(72) Inventors: James W. Hodge, Kensington, MD (US); Sofia R. Gameiro, North Bethesda, MD (US)

(73) Assignee: The United States of America, as represented by the Secretary, Dept. of Health and Human Services, Bethesda, MD (US)

(21) Appl. No.: 16/066,693

(22) PCT Filed: Jan. 4, 2017
(86) PCT No.: PCT/US2017/012149

§ 371 Date: Jun. 28, 2018

Related U.S. Application Data
(60) Provisional application No. 62/274,946, filed on Jan. 5, 2016.
(60) Provisional application No. 62/278,852, filed on Jan. 14, 2016.

(51) Int. Cl. A61K 31/4406; A61K 31/167; A61P 35/00; A61K 31/4166; A61K 31/58; A61K 39/00; A61K 39/235; A61K 39/275

(52) U.S. Cl. CPC A61K 31/4406; A61K 31/167; A61P 35/00; A61K 31/4166; A61K 31/58; A61K 39/001102; A61K 39/235; A61K 39/001152; A61K 39/275; A61K 39/001182; A61K 39/001194

(57) ABSTRACT

A method of reducing cancer cell growth, a method of increasing sensitivity of cancer cells to CTL mediated killing, and a method of increasing sensitivity of cancer cells to NK mediated killing are provided. The methods comprise treating cancer cells with a combination of a HDAC inhibitor and immunotherapy.

### CROSS-REFERENCE TO RELATED APPLICATIONS

This patent application claims the benefit of U.S. Provisional Patent Application No. 62/274,946, filed Jan. 5, 2016, and U.S. Provisional Patent Application No. 62/278,852, filed Jan. 14, 2016, each of which is incorporated by reference.

### SEQUENCE LISTING

Incorporated by reference in its entirety herein is a nucleotide/amino acid sequence listing submitted concurrently herewith.

### BACKGROUND OF THE INVENTION

Mounting evidence suggests that evasion of host immune surveillance is a key determinant of tumor progression (Rooney et al., Cell, 160(1-2): 48-61 (2015); Hicklin et al., Mol. Med. Today, 5(4): 178-186 (1999); and Johnsen et al., J. Immunol., 163(8): 4224-4231 (1999)). Immune evasion also is a major obstacle to the efficacy of cancer immunotherapies, therefore preventing long-lasting tumor control.

Multiple strategies have been investigated to improve immune recognition of malignant tumors (Ardiani et al., Oncotarget, 5(19): 9335-9348 (2014); Del Campo et al., Cancer Gene Ther., 21(8): 317-332; and Hodge et al., Int. J. Cancer, 133(3): 624-636 (2013)). Recent evidence suggests that certain anticancer therapies can alter the biology of the surviving cell population to restore their sensitivity to T-cell-mediated lysis (Ardiani et al., Oncotarget, 5(19): 9335-9348 (2014); Hodge et al., Int. J. Cancer, 133(3): 624-636 (2013); and Gameiro et al., Oncoimmunology, 3: e28643 (2014)). Mechanistic examination of this reversal of tumor immune evasion, also known as immunogenic modulation, determined it to be a consequence of a spectrum of biological adaptations to cellular stress, resulting in enhanced antigen processing and augmented tumor recognition (Hodge et al., Int. J. Cancer, 133(3): 624-636 (2013); Gameiro et al., Oncoimmunology, 3: e28643 (2014); and Gameiro et al., Oncotarget, 5(2): 403-416 (2014)). Strong evidence also has implicated tumor epigenetic silencing of immune-associated genes as a determinant of an immune evasion signature (Wrangle et al., Oncotarget, 4(11): 2067-2079 (2013); Hellebrekers et al., Cancer. Res., 66(22): 10770-10777 (2006); and Choudhary et al., Science, 325(5942): 834-840 (2009)). Epigenetic deregulation has been associated with worse prognosis in a wide spectrum of malignancies, including lung, breast and prostate (West et al., J. Clin. Invest., 124(1): 30-39 (2014); Burdelski et al., Exp. Mol. Pathol., 98(3): 419-426 (2015); and Muller et al., BMC Cancer, 13: 215 (2013)). Epigenetic silencing can occur at multiple levels, with DNA methylation and chromatin deacetylation having been identified as two major determinants (Choudhary et al., Science, 325(5942): 834-840 (2009); and Campoli et al., Oncogene, 27(45): 5869-5885 (2008)). Unlike other types of malignant deregulation, such as oncogenic mutations, epigenetic alterations are mostly reversible, offering an exceptional therapeutic opportunity. However, despite its worth for the treatment of hematological malignancies, the promise of epigenetic therapy has not been realized for solid malignancies, albeit encouraging reports (Azad et al., Nat. Rev. Clin. Oncol., 10(5): 256-266 (2013); and Juergens et al., Cancer Discov., 1(7): 598-607 (2011)). Strong evidence from the last decade of clinical experience in the treatment of solid tumors with epigenetic agents strongly supports their use in combination with therapeutic modalities that can capitalize on the broad spectrum of tumor epigenetic reprogramming that they induce (Azad et al., Nat. Rev. Clin. Oncol., 10(5): 256-266 (2013)). On this basis, multiple clinical studies have shown promising clinical activity in the management of solid malignancies when combining inhibitors of DNA methyltransferases (DNMT) or histone deacetylases (HDACs), including vorinostat and entinostat, with cytotoxic agents (Azad et al., Nat. Rev. Clin. Oncol., 10(5): 256-266 (2013); and Ahuja et al., J. Clin. Invest., 124(1): 56-63 (2014)).

Vorinostat is an orally bioavailable hydroxamate pan-HDAC inhibitor currently approved in the United States for the treatment of cutaneous T-cell lymphoma (West et al., J. Clin. Invest., 124(1): 30-39 (2014)). Vorinostat inhibits a broad spectrum of HDAC enzymes, namely class I (HDACs 1 to 3), and class IIb (HDACs 6 and 10), whereas entinostat specifically inhibits class I HDAC enzymes (HDACs 1 to 3, and 8) (West et al., J. Clin. Invest., 124(1): 30-39 (2014)). Both agents have shown synergistic antitumor activity in combination with checkpoint inhibitors and agonistic antibodies in murine models of solid malignancies (Kim et al., Proc. Natl. Acad. Sci. USA, 111(32): 11774-11779 (2014); and Christiansen et al., Proc. Natl. Acad. Sci. USA, 108(10): 4141-4146 (2011)). This synergy is in agreement with particular characteristics of these agents, including induction of immunogenic cell death by vorinostat, and suppression of tumor-initiating cells, regulatory T cells, and myeloid-derived suppressor cells by entinostat (Schech et al., Mol. Cancer Ther., 14(8): 1848-1857 (2015); Sigalotti et al., Pharmacol. Ther., 142(3): 339-350 (2014); and Pili et al., Br. J. Cancer, 106(1): 77-84 (2012)).

In a recent clinical report in which advanced stage, heavily pretreated non-small cell lung cancer (NSCLC) patients were treated with entinostat and the DNMT inhibitor azacitidine, 4 out of 19 patients showed major objective responses to subsequent anticancer therapies given immediately after epigenetic therapy, including immunotherapy targeting the checkpoint inhibitor PD1. Subsequent in vitro studies in NSCLC cell lines indicated that azacitidine induced an expression signature of immune genes and pathways (Wrangle et al., Oncotarget, 4(11): 2067-2079 (2013)), suggesting that epigenetic therapy of solid tumors may reprogram the tumor to reverse its immune evasion signature, thus priming it for a more efficient immune attack. This concept is further supported by in vivo and in vitro preclinical studies with HDAC inhibitors (Sigalotti et al., Pharmacol. Ther., 142(3): 339-350 (2014); and Setiadi et al., Cancer Res., 68(23): 9601-9607 (2008)). However, findings on the effect of epigenetic modulation of immune genes in human carcinoma cell lines have been contradictory (Woan et al., Immunol. Cell Biol., 90(1): 55-65 (2012); Pellicciotta et al., Cancer Res., 68(19): 8085-8093 (2008); and Fiegler et al., Blood, 122(5): 684-693 (2013)). These discrepancies may be the result of tumor type inherent expression of specific HDAC enzymes as well as a consequence of very distinct and non-clinically observed drug overexposures used, potentially translating into a multitude of non-target effects.

Hence, there is an unmet clinical need to develop effective therapeutic strategies to restore tumor immune recognition and promote long-lasting tumor control, which can be further augmented when combined with immunotherapy, such as immune checkpoint blockade or therapeutic cancer vaccines.

### BRIEF SUMMARY OF THE INVENTION

The invention provides a method of reducing cancer cell growth, which method comprises treating cancer cells with a combination of a histone deacetylase (HDAC) inhibitor and immunotherapy, whereupon growth of the cancer cells is reduced.

The invention also provides a method of increasing sensitivity of cancer cells to cytotoxic T-cell (CTL) mediated killing, which method comprises treating cancer cells with a combination of a HDAC inhibitor and immunotherapy, whereupon the sensitivity of the cancer cells to CTL mediated killing is increased.

The invention further provides a method of increasing sensitivity of cancer cells to natural killer (NK) cell mediated killing, which method comprises treating cancer cells with a combination of a HDAC inhibitor and immunotherapy, whereupon the sensitivity of the cancer cells to NK mediated killing is increased.

### BRIEF DESCRIPTION OF THE SEVERAL VIEWS OF THE DRAWINGS

FIG. 1A-D are graphs demonstrating that vorinostat decreases pan-HDAC activity and proliferation of human carcinoma cells in an exposure-dependent manner. Human prostate (LNCaP) (FIGS. 1A and 1C) and breast (MDA-MB-231) (FIGS. 1B and 1D) carcinoma cells were exposed to vorinostat (1 μM, grey circles and bars; 3 μM, black circles and bars), or vehicle (DMSO, open squares and bars). For FIG. 1A-B, HDAC activity was determined at 96 h, and results presented as mean±S.E.M. from replicate wells. For FIG. 1C-D, cell number at the indicated time points was determined. Insets denote viability at 96 h. Results are presented as mean±S.D. from 6 replicate wells. Asterisks denote statistical significance relative to control cells exposed to vehicle (DMSO, P<0.001). This experiment was repeated 2-3 times with similar results.

FIG. 2 is a series of graphs showing that carcinoma cells exposed to vorinostat are significantly more sensitive to cyotoxic T-cell (CTL)-mediated killing. Human prostate (LNCaP) and breast (MDA-MB-231) carcinoma cells were exposed to vorinostat (3 μM, black bars) or to vehicle (DMSO, open bars) prior to being used as targets for antigen-specific CTL lysis using CEA-, brachyury-, MUC-1-, or PSA-specific CD8⁺ T cells as effector cells (E:T=30:1). To verify that effector T cells were HLA-restricted, CTLs were incubated with HLA-A2 negative AsPC-1 pancreatic carcinoma cells exposed to vehicle (DMSO) or vorinostat. Results are presented as mean±S.E.M. from 3-6 replicate wells, and are representative of 1-4 independent experiments. Asterisks denote statistical significance relative to controls.

FIG. 3 is a table showing the effect of vorinostat on protein expression of antigen processing machinery (APM) components in human breast carcinoma cells. MDA-MB-231 cells were exposed to vorinostat (3 μM) or vehicle (DMSO) control. At the end of treatment (96 h), cells were analyzed by flow cytometry for cellular expression of indicated APM components. Bold denotes significant modulation (≥25% change in percent of cells or mean fluorescence intensity (MFI) not observed in isotype control vs. untreated cells).

FIG. 4A-B are graphs showing vorinostat-induced immunogenic modulation of MDA-MB-231 carcinoma cells is mediated by HDAC1. MDA-MB-231 cells were exposed to silencing RNA (siRNA) control or targeting HDAC1 for 24 h prior to being exposed to vehicle (DMSO) or vorinostat (3 μM). For FIG. 4A, total cell lysates were examined by Western blotting to determine expression of HDAC1 at the end of treatment. GAPDH was used as internal control for total protein levels. Silencing ratio denotes HDAC1 protein expression relative to GAPDH, further normalized to protein levels after treatment in the presence of silencing RNA control. For FIG. 4B, at the end of treatment, MDA-MB-231 cells were used as targets in a CTL-lysis assay where effector brachyury-specific CD8⁺ T cells were used at an E:T ratio of 30:1. Results are presented as mean±S.E.M. from 4-6 replicate wells. Asterisks denote statistical significance relative to controls (*P=0.002). Data is representative of two independent experiments.

FIG. 5A-C demonstrates that HDAC inhibition activates the endoplasmic reticulum (ER) stress responsive element in LNCaP carcinoma cells in a dose-dependent manner. For FIG. 5A, single-cell clones of LNCaP cells stably transduced with an ER stress responsive element driving firefly luciferase expression were exposed to vorinostat or entinostat at the designated concentrations or DMSO controls. At the end of treatment, firefly and renilla luciferase activities were determined. Data are shown as the ratio of firefly luciferase activity relative to that of control renilla luciferase within each well, further normalized to DMSO control. Results are presented as mean±S.E.M. from 4-6 replicate wells, and are representative of two independent experiments. For FIG. 5B, parental LNCaP prostate carcinoma cells were exposed to vorinostat (3 μM), entinostat (500 nM) or to vehicle (DMSO) controls prior to being used as targets for antigen-specific CTL lysis using PSA-specific CD8⁺ T cells as effector cells (E:T=30:1). Results are presented as mean±S.E.M. from 6 replicate wells. Asterisks denote statistical significance relative to controls (P<0.05). FIG. 5C is a schematic representation of immunogenic modulation induced by HDAC inhibition in human carcinoma cells.

FIG. 6A-D show vorinostat-induced immunogenic modulation is mediated by the unfolded protein response. MDA-MB-231 cells were exposed to siRNA control or targeting endoplasmic reticulum to nucleus signaling 1 (ERN1) or protein kinase R (PKR-like endoplasmic reticulum kinase (PERK) for 24 h prior to being exposed to vehicle (DMSO) or vorinostat (3 μM). For FIG. 6A-B, at the end of treatment, total cell lysates were examined by Western blotting to determine expression of ERN1 (FIG. 6A) or PERK (FIG. 6B). GAPDH was used as internal control for total protein levels. Silencing ratio denotes target protein expression relative to GAPDH, further normalized to protein levels after treatment in the presence of silencing RNA control. For FIG. 6C-D, at the end of treatment, MDA-MB-231 cells were used as targets in a CTL lysis assay using CEA-specific CD8⁺ T cells as effectors (E:T=30:1). Results are presented as mean±S.E.M. from 6 replicate wells, and are representative of 2-3 independent experiments. Asterisks denote statistical significance relative to controls (P<0.0001).

FIG. 7 is a series of tables and graphs showing the effect of vorinostat on the sensitivity of human prostate (LNCaP), breast (MDA-MB-231), or lung (H460) carcinoma cells to human NK killing. Carcinoma cells were exposed to vorinostat (3 μM, closed circles) or to vehicle (DMSO, open circles) prior to being used as targets for human NK lysis at indicated effector:target (E:T) ratios in a standard overnight cytotoxicity ¹¹¹In-release assay. In the upper tables, LNCaP, MDA-MB-231, and H460 MIC A/B cell-surface expression was determined by flow cytometry upon exposure to vorinostat or DMSO control. Numbers denote percentage of cells expressing MICA/B on the cell surface with MFI in parenthesis. Bold denotes an expression increase above 30% relative upon exposure to vorinostat relative to that of cells exposed to DMSO control.

FIG. 8 is a series of tables and graphs demonstrating that vorinostat increases avelumab-mediated ADCC in human lung (H460) carcinoma cells. Lung (H460) and pancreatic (AsPC-1) carcinoma cells were exposed daily for 5 h to vorinostat (3 uM, black circles and bars) or DMSO (open circles and bars) for 4 consecutive days prior to being used as targets PDL-1-mediated ADCC. Upper panel: cell-surface expression of PDL1 in carcinoma targets. Middle panel: NK lysis in the presence of anti-PDL1 or isotype control Ab. Lower panel: H460 lysis in the presence of anti-PDL1 or isotype control antibodies using NK effector cells pre-incubated with anti-CD16 mAb. Results are presented as mean±S.E.M. from 3 replicate wells and are representative of 2-4 independent experiments. Asterisks denote statistical significance relative to controls (2-way ANOVA).

### DETAILED DESCRIPTION OF THE INVENTION

The invention is predicated, at least in part, on the discovery that clinically relevant exposure of prostate and breast human carcinoma cells to histone deacetylase (HDAC) inhibitors reverses tumor immune escape to T-cell mediated lysis. Prostate and breast carcinoma cells are more sensitive to T-cell and NK cell mediated lysis in vitro after clinically relevant exposure to epigenetic therapeutic agents targeting HDAC (e.g., the pan-HDAC inhibitor vorinostat or the class 1 HDAC inhibitor entinostat). HDAC inhibition also was shown to upregulate the Programmed cell death 1 ligand 1 (PD-L1) on tumor cells and increase sensitivity to anti-PD-L1 mediated ADCC (sensitivity to NK mediated killing). This pattern of immunogenic modulation was observed against a broad range of tumor-associated antigens (TAAs), such as carcinoembryonic antigen (CEA), mucin-1 (MUC-1), prostate-specific antigen (PSA), and brachyury, and associated with augmented expression of multiple proteins involved in antigen processing and tumor immune recognition. Genetic and pharmacological inhibition studies identified HDAC1 as a key determinant in the reversal of carcinoma immune escape. Although not wishing to be bound by any particular theory, it appears that the observed reversal of epigenetic silencing promoting immune evasion is driven by a response to cellular stress through activation of the unfolded protein response (UPR).

Therefore, the invention provides a method of reducing cancer cell growth, which method comprises treating cancer cells with a combination of a HDAC inhibitor and immunotherapy, whereupon growth of the cancer cells is reduced.

Additionally, the invention provides a method of increasing sensitivity of cancer cells to cytotoxic T-cell (CTL) mediated killing, which method comprises treating cancer cells with a combination of a HDAC inhibitor and immunotherapy, whereupon the sensitivity of the cancer cells to CTL mediated killing is increased.

The invention further provides a method of increasing sensitivity of cancer cells to natural killer (NK) cell mediated killing, which method comprises treating cancer cells with a combination of a HDAC inhibitor and immunotherapy, whereupon the sensitivity of the cancer cells to NK mediated killing is increased (i.e., antibody dependent cell mediated cytotoxicity (ADCC) by NK cells is increased).

Non-limiting examples of specific types of cancers include cancer of the head and neck, eye, skin, mouth, throat, esophagus, chest, bone, lung, colon, sigmoid, rectum, stomach, prostate, breast, ovaries, kidney, liver, pancreas, brain, intestine, heart or adrenals. More particularly, cancers include solid tumor, sarcoma, carcinomas, fibrosarcoma, myxosarcoma, liposarcoma, chondrosarcoma, osteogenic sarcoma, chordoma, angiosarcoma, endotheliosarcoma, lymphangiosarcoma, lymphangioendothelio sarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, Kaposi's sarcoma, pinealoma, hemangioblastoma, acoustic neuroma, oligodendroglioma, menangioma, melanoma, neuroblastoma, retinoblastoma, a blood-born tumor, acute lymphoblastic leukemia, acute lymphoblastic B-cell leukemia, acute lymphoblastic T-cell leukemia, acute myeloblastic leukemia, acute promyelocytic leukemia, acute monoblastic leukemia, acute erythroleukemic leukemia, acute megakaryoblastic leukemia, acute myelomonocytic leukemia, acutenonlymphocyctic leukemia, acute undifferentiated leukemia, chronic myelocytic leukemia, chronic lymphocytic leukemia, hairy cell leukemia, or multiple myeloma. See, e.g., Harrison's Principles of Internal Medicine, Eugene Braunwald et al., eds., pp. 491 762 (15th ed. 2001).

In one embodiment, the cancer is prostate cancer. The term “prostate cancer,” which is also synonymous with the term “prostate carcinoma,” refers to cancer that forms in tissues of the prostate. “Prostate cancer cells” refer to cells obtained or derived from a prostate cancer. In another embodiment, the inventive method can be used to inhibit growth of hyperplastic, but not malignant, prostate cells, such as, for example, high grade prostatic intraepithelial neoplasia (HGPIN) or benign prostatic hyperplasia (BPH), which is also referred to in the art as benign enlargement of the prostate (BEP), adenofibromyomatous hyperplasia, and benign prostatic hypertrophy.

The prostate cancer cells can be of any grade or stage, as determined by histopathology and the Gleason score, and/or in accordance with the guidelines described in, e.g., Edge et al. (eds.), American Joint Committee on Cancer (AJCC) Staging Manual, 7^{th} Edition (2010), or the SEER Program Coding and Staging Manual, NIH Publication Number13-5581, U.S. Department of Health and Human Services National Cancer Institute (2013).

The prostate cancer cells can have been subjected to one or more prostate cancer therapies (e.g., surgery, chemotherapy, androgen deprivation therapy, and/or radiation) prior to the inventive method. In this respect, most hormone-dependent prostate cancers become refractory to androgen deprivation therapy after one to three years and resume growth despite androgen deprivation therapy. Such cancers are known as castration resistant prostate cancer (CRPC). The prostate cancer cells can be metastatic castration resistant prostate cancer cells, which are resistant to treatment with androgen deprivation therapy alone. In another embodiment, the prostate cancer cells have become resistant to other standard treatment regimens. For example, the prostate cancer cells can be resistant to chemotherapy and/or radiation therapy.

The prostate cancer cells can express an androgen receptor (AR). The androgen receptor (AR), also known as NR3C4 (nuclear receptor subfamily 3, group C, member 4), is a nuclear receptor that is activated by binding of either of the androgenic hormones testosterone or dihydrotestosterone in the cytoplasm, and is translocated into the nucleus where it functions as a DNA-binding transcription factor (Roy et al., Vitamins & Hormones, 55: 309-352 (1999)). AR signaling plays a critical role in the development, function, and homeostasis of the prostate. Prostate cancer initiation and progression also is dependent on AR (Lonergan P E, Tindall D J., J. Carcinog., 10: 20 (2011)). AR expression is maintained throughout prostate cancer progression, and the majority of androgen-independent or hormone refractory prostate cancers express AR. Mutation of AR may contribute to the progression of prostate cancer and the failure of endocrine therapy by allowing AR transcriptional activation in response to antiandrogens or other endogenous hormones (Heinlein and Chang, Endocr. Rev., 25(2): 276-308 (2004)). AR also is widely expressed in breast cancers and has been proposed as a therapeutic target in estrogen-receptor (ER) negative breast cancers that express AR (Cochrane et al., Breast Cancer Res., 16: R7 (2014)).

In another embodiment, the cancer is breast cancer. The term “breast cancer” is synonymous with the term “breast carcinoma,” and refers to cancer that forms in tissues of the breast or mammary gland. “Breast cancer cells” refer to cells obtained or derived from a breast cancer. In another embodiment, the inventive method can be used to inhibit growth of hyperplastic, but not malignant, breast cells, such as, for example, usual hyperplasia or atypical hyperplasia.

The breast cancer cells also can be of any grade or stage, as determined by a variety of factors including tumor size, lymph node status, estrogen-receptor and progesterone-receptor levels in the tumor tissue, human epidermal growth factor receptor 2 (HER2/neu) status, menopausal status, and the general health of the patient. Cancer staging and grading guidelines are described in detail in, e.g., Edge et al. (eds.), American Joint Committee on Cancer (AJCC) Staging Manual, 7^{th} Edition (2010), or the SEER Program Coding and Staging Manual, NIH Publication Number13-5581, U.S. Department of Health and Human Services National Cancer Institute (2013).

The breast cancer cells can have been subjected to one or more breast cancer therapies (e.g., surgery, chemotherapy, and/or radiation) prior to the inventive method. In another embodiment, the breast cancer cells have become resistant to other standard treatment regimens. For example, the breast cancer cells can be resistant to chemotherapy and/or radiation therapy.

The breast cancer cells can be positive or negative for an androgen receptor (AR). As discussed above, AR is widely expressed in breast cancers and has been proposed as a therapeutic target in estrogen-receptor (ER) negative breast cancers that express AR (Cochrane et al., Breast Cancer Res., 16: R7 (2014)). The breast cancer cells can express an androgen receptor. Alternatively, the breast cancer cells do not express an androgen receptor. The breast cancer cells also can be positive or negative for an estrogen receptor (ER). The estrogen receptor is a ligand-activated transcription factor composed of several domains that are important for hormone binding, DNA binding, and activation of transcription. The ER is activated by 17β-estradiol, and binding of estrogen to the ER stimulates proliferation of mammary cells. The estrogen receptor is overexpressed in about 70% of breast cancers (referred to as “ER-positive” breast cancers). In one embodiment, the breast cancer cells express an estrogen receptor. Alternatively, the breast cancer cells do not express an estrogen receptor.

The term “immunotherapy,” as used herein refers to the treatment of a disease by inducing, enhancing, or suppressing an immune response. Immunotherapies designed to elicit or enhance an immune response are referred to as activation immunotherapies, while immunotherapies designed to suppress an immune response are referred to suppression immunotherapies. Types of immunotherapies include, but are not limited to, checkpoint inhibitors, immunomodulators, cell-based immunotherapies, monoclonal antibodies, radiopharmaceuticals, and vaccines. Immunotherapy strategies for cancer are described in, for example, Waldmann, T. A., Nature Medicine, 9: 269-277 (2003).

Immunomodulators can be recombinant, synthetic, or natural substances that include, but are not limited to, cytokines (e.g., TNF-α, IL-6, GM-CSF, IL-2, and interferons), co-stimulatory molecules (e.g., B7-1 and B7-2), chemokines (e.g., CCL3, CCL26, CXCL7), glucans, and oligodeoxynucleotides.

Cell-based immunotherapies typically involve removal of immune cells (e.g., cytotoxic T-cells, natural killer cells, or antigen presenting cells (APCs)) from a subject, modification (e.g., activation) of immune cells, and return of the modified immune cells to the patient. In the context of the inventive method, the cell-based immunotherapy desirably is Sipuleucel-T (PROVENGE™), which is an autologous active cellular immunotherapy used in the treatment of asymptomatic or minimally symptomatic CRPC (Plosker, G. L., Drugs, 71(1): 101-108 (2011); and Kantoff et al., New Engl. J. Med., 363: 411-422 (2010)).

Several monoclonal antibodies have been approved for the treatment of cancer, including naked antibodies and antibody-drug conjugates based on human, humanized, or chimeric antibodies (Scott et al., Nat Rev Cancer, 12(4): 278-87 (2012); Harding et al., MAbs, 2(3): 256-65 (2010); and Weiner et al., Nature Rev. Immunol., 10(5): 317-327 (2010)). In one embodiment, the inventive method comprises treating the prostate cancer cells with any suitable monoclonal antibody known in the art. Such monoclonal antibodies include, for example, ipilumimab (YERVOY™), which is a fully human antibody that binds to CTLA-4 and is indicated for the treatment of melanoma. Antibodies that target the interaction of programmed death receptor-1 (PD-1) with its ligands PD-L1 and PD-L2, also can be used in the invention (see, e.g., Weber, Semin. Oncol., 37(5): 430-4309 (2010); and Tang et al., Current Oncology Reports, 15(2): 98-104 (2013)). Antibodies that inhibit PD-1 signaling include, for example nivolumab (also known as BMS-936558 or MDX1106; see, e.g., ClinicalTrials.gov Identifier NCT00730639), sipuleucel-T CT-011, pembrolizumab, atezolizumab, and MK-3575 (see, e.g., Patnaik et al., 2012 American Society of Clinical Oncology (ASCO) Annual Meeting, Abstract #2512). Monoclonal antibodies that specifically target prostate cancer are under development and also can be used in the invention (see, e.g., Jakobovits, A., Handb. Exp. Pharmacol., 181: 237-56 (2008); and Ross et al., Cancer Metastasis Rev., 24(4): 521-37 (2005)). Monoclonal antibodies suitable for treatment of breast cancer include, for example, trastuzumab (HERCEPTIN™), pertuzumab (PERJETA™), and the antibody-drug conjugate ado-trastuzumab emtansine (KADCYLA™).

Radiopharmaceuticals are radioactive drugs which are currently used to treat and diagnose a variety of diseases, including cancer. For example, radionuclides can be targeted to antibodies (i.e., radioimmunotherapy) to treat blood-derived cancers (Sharkey, R. M. and Goldenberg, D. M., Immunotherapy, 3(3): 349-70 (2011)). Several radioisotopes have been approved to treat cancer, including iodine-125, iodine-131, and radium-223 (marketed as XOFIGO™). Radium-223 has been approved as a radiopharmaceutical to treat metastatic bone cancer and CRPC. In CRPC, radium-223 also has been shown to enhance the antitumor immune response.

Vaccines represent another strategy to prevent and treat cancer. Many different cancer vaccine platforms are currently being evaluated in phase II and/or phase III clinical trials, including, for example, peptide-based vaccines, recombinant viral vectors, killed tumor cells, or protein-activated dendritic cells (see, e.g., Schlom, J., J. Natl. Cancer. Inst., 104: 599-613 (2012)). Any suitable vaccine can be used in the inventive method.

In one embodiment, the vaccine is a virus-based vaccine, such as a poxviral-based or adenoviral-based vaccine. For example, the vaccine can be the PSA/TRICOM vaccine (PROSTVAC™), which is a cancer vaccine composed of a series of poxviral vectors engineered to express PSA and a triad of human T-cell costimulatory molecules (see, e.g., Madan et al., Expert Opin. Investigational Drugs, 18(7): 1001-1011 (2009); and U.S. Pat. Nos. 4,547,773; 6,045,802; 6,165,4,60; 6,548,068; 6,946,133; 7,247,615; 7,368,116; 7,598,225; 7,662,395; 7,871,986; and 8,178,508). The vaccine also can be a MUC-1/CEA vaccine (e.g., PANVAC), which is composed of a series of poxviral vectors (e.g., recombinant vaccinia and recombinant fowlpox) engineered to express MUC-1 and CEA and optionally human T-cell costimulatory molecules (e.g., TRICOM) (see, e.g., Madan et al., Expert Opin Biol Ther., 7(4): 543-54; International Patent Application Publications WO 2005/046622, WO 2005/046614, and WO 2015/061415); and U.S. Pat. Nos. 5,698,530; 6,001,349; 6,319,496; 6,969,609; 7,211,432; 7,368,116; 7,410,644; 7,771,715; 7,999,071; and 8,609,395). Alternatively, the cancer vaccine can comprise poxviral vectors (e.g., MVA and/or fowlpox) that have been genetically modified to express CEA and TRICOM (e.g., MVA/rF-CEA/TRICOM). The vaccine also can be a yeast MUC-1 immunotherapeutic, such as those described in, e.g., U.S. Patent Application Publication 2013/0315941 and International Patent Application Publication WO 2012/103658.

In another embodiment, the vaccine can be a Brachyury vaccine, which comprises recombinant yeast or poxvirus that has been genetically modified to express the Brachyury transcription factor (see, e.g., International Patent Application Publications WO 2014/043518 and WO 2014/043535; and U.S. Pat. Nos. 8,188,214 and 8,613,933).

Any suitable HDAC inhibitor can be used in the methods described herein. Exemplary HDAC inhibitors include, but are not limited to, hydroxamates (e.g., TSA, vorinostat, M-Carboxycinnamic acid bishydroxamate (CBHA) and derivatives thereof (e.g., LAQ-824, belinostat (PDX-101), and Panobinostat (LBH-589)), ITF2357 (Italfarmaco SpA), and PC1-24781), cyclic peptides (e.g., depsipeptide (FK-228), apicidin, and the cyclic hydroxamic acid-containing peptide group of molecules), aliphatic acids (valproic acid, phenyl butyrate, butyrate, and pivaloyloxymethyl butyrate (AN-9)), and benzamides or derivatives thereof (5 NOX-275 (MS-275), MGCD0103, and entinostat) (Dokmanovic, Mol. Cancer Res., 5: 981 (2007)). In one embodiment, the HDAC inhibitor is selected from the group consisting of apcidin, belinostat, entinostat, mocetinostat, panobinostat, abexinostat, PC1-334051, romidepsin, vorinostat, trichostatin A, and valproic acid (West et al, J. Clin. Invest. 124(1): 30-39 (2014)).

Preferably, the HDAC inhibitor is a HDAC inhibitor is a class I HDAC inhibitor. Exemplary, class I HDAC inhibitors include apcidin, belinostat, entinostat, mocetinostat, panobinostat, abexinostat, romidepsin, vorinostat, trichostatin A, and valproic acid. In one embodiment, the HDAC inhibitor is vorinostat or entinostat.

The combination of immunotherapy and a HDAC inhibitor reduces or inhibits growth of cancer cells (e.g., prostate cancer cells, breast cancer cells, lung cancer cells, or colon cancer cells). The term “growth,” as used herein, encompasses any aspect of the growth, proliferation, and progression of cancer cells, including, for example, cell division (i.e., mitosis), cell growth (e.g. increase in cell size), an increase in genetic material (e.g., prior to cell division), and metastasis. Reduction, inhibition, or suppression of cancer cell growth includes, but is not limited to, inhibition of cancer cell growth as compared to the growth of untreated or mock treated cells, inhibition of proliferation, inhibition of metastases, sensitization to immune-mediated killing (e.g., T-cell-mediated lysis), induction of cancer cell senescence, induction of cancer cell death, and reduction of tumor size.

The cancer cells (e.g., prostate cancer cells, breast cancer cells, lung cancer cells, or colon cancer cells) can be in vivo or in vitro. The term “in vivo” refers to a method that is conducted within living organisms in their normal, intact state, while an “in vitro” method is conducted using components of an organism that have been isolated from its usual biological context (e.g., isolating and culturing cells obtained from an organism). Preferably, the cancer cells are in vivo. For example, when the cancer cells are prostate cancer cells, preferably the prostate cancer cells exist within a human male prostate cancer patient. When the cancer cells are breast cancer cells, preferably the breast cancer cells exist within a human male or female breast cancer patient. When the cancer cells (e.g., prostate cancer cells, breast cancer cells, lung cancer cells, or colon cancer cells) are in vivo, i.e., in a human, the inventive methods induce a therapeutic effect in the cancer patient and treat the cancer (e.g., prostate cancer, breast cancer, lung cancer, or colon cancer).

The patient can be any suitable patient, such as a mammal (e.g., mouse, rat, guinea pig, hamster, rabbit, cat, dog, pig, goat, cow, horse, or primate (e.g., human)).

As used herein, the terms “treatment,” “treating,” and the like refer to obtaining a desired pharmacologic and/or physiologic effect. Preferably, the effect is therapeutic, i.e., the effect partially or completely cures a disease and/or adverse symptom attributable to the disease. To this end, the inventive method comprises administering a “therapeutically effective amount” of the immunotherapy and a HDAC inhibitor. A “therapeutically effective amount” refers to an amount effective, at dosages and for periods of time necessary, to achieve a desired therapeutic result. The therapeutically effective amount may vary according to factors such as the disease state, age, and weight of the individual, and the ability of the immunotherapy and the HDAC inhibitor to elicit a desired response in the individual.

The combination of a HDAC inhibitor and immunotherapeutic agent (e.g., cancer vaccine) can be administered sequentially or simultaneously. In certain embodiments, one or more (e.g., 2, 3, 4, or 5) HDAC inhibitors is administered in combination with one or more (e.g., 2, 3, 4, or 5) immunotherapeutic agents (e.g., cancer vaccines). In additional embodiments, the combination of a HDAC inhibitor and immunotherapeutic agent can be administered with one or more (e.g., 2, 3, 4, or 5) additional therapeutic agents (e.g., endocrine deprivation therapy, androgen deprivation therapy, and/or cabozantinib).

The term “androgen deprivation therapy” (ADT), as used herein, refers to a treatment for cancer in which the level of androgen hormones, such as testosterone, in a patient are reduced, typically by pharmaceutical or surgical methods (see, e.g., Perlmutter and Lepor, Rev. Urol., 9 (Suppl 1): S3-8 (2007)). Surgical approaches to ADT include surgical castration. Pharmaceutical approaches to ADT include androgen inhibitors (antiandrogens) and chemical castration. ADT also is referred to in the art as androgen suppression therapy. Androgen inhibitors used in prostate cancer can be steroidal or non-steroidal (also referred to as “pure” antiandrogens). Steroidal androgen inhibitors include, for example, e.g., megestrol (MEGACE™), cyproterone acetate, abiraterone, and abiraterone acetate (ZYTIGA™). Non-steroidal androgen inhibitors include, for example, bicalutamide (CASODEX™), flutamide (EULEXIN™), nilutamide (ANANDRON™ and NILANDRON™), and enzalutamide (XTANDI™).

In one embodiment, the androgen deprivation therapy is enzalutamide. Enzalutamide (marketed as XTANDI™ by Medivation and Astellas and formally known as MDV3100) is an oral non-steroidal small molecule androgen receptor inhibitor that prolongs survival in men with metastatic castration resistant prostate cancer in whom the disease has progressed after chemotherapy. Preclinical studies also suggest that enzalutamide also inhibits breast cancer cell growth (see, e.g., Cochrane et al., Cancer Research, 72(24 Suppl): Abstract nr P2-14-02 (2012)).

Immunogenic modulation by enzalutamide has been described in murine prostate carcinomas (see, e.g., Ardiani et al., Clinical Cancer Res., 19(22): 6205-6218 (2013)), where enzalutamide up-regulated MHC-I and Fas on the surface of tumor cells, thus improving the cells' sensitivity to T-cell killing. In these studies, treatment with enzalutamide did not alter the number or function of T-cells. Enzalutamide-mediated immunogenic modulation increased the efficacy of a therapeutic cancer vaccine in TRAMP mice with spontaneous prostate tumors, which subsequently translated to significant improvements in overall survival (Ardiani et al., supra).

In another embodiment, the androgen deprivation therapy is abiraterone, which is formulated as abiraterone acetate and marketed as ZYTIGA™ by Janssen Biotech, Inc. Abiraterone inhibits CYP17A1, a rate-limiting enzyme in androgen biosynthesis. Inhibition of CYP17A1 subsequently blocks the production of androgen in all endocrine organs, including the testes, adrenal glands, and in prostate tumors (Harris et al., Nature Clinical Practice Urology, 6(2): 76-85 (2009)). In a phase III study in patients with CRPC previously treated with docetaxel, abiraterone was shown to improve overall survival by 3.9 months compared to placebo (de Bono et al., New England J. Med., 364(21): 1995-2005 (2011)). Abiraterone is indicated for use in combination with prednisone to treat CRPC.

The term “endocrine deprivation therapy” (also referred to as “hormonal therapy”), as used herein, refers to a treatment for breast cancer in which the level of endocrine hormones, such as estrogen and/or testosterone, in a patient are reduced, typically by pharmaceutical or surgical methods (see, e.g., Angelopoulos et al., Endocr. Relat. Cancer, 11: 523-535 (2004); Dhingra, K., Invest. New Drugs, 17(3): 285-311 (1999); and Garay, J. P. and Park, B. H., Am. J. Cancer Res., 2(4): 434-445 (2012)). Surgical approaches to endocrine deprivation include oophorectomy. Pharmaceutical approaches to endocrine deprivation therapy include estrogen inhibitors and androgen inhibitors. In one embodiment, the endocrine deprivation therapy is an androgen inhibitor such as, for example, cyproterone acetate, abiraterone, abiraterone acetate (ZYTIGA™), or enzalutamide (XTANDI™). The androgen inhibitor preferably is abiraterone or enzalutamide. Alternatively or additionally, the endocrine deprivation therapy is an estrogen inhibitor, such as, for example, megestrol (MEGACE™), an aromatase inhibitor (e.g., anastrozole), a selective estrogen receptor down-regulator (SERD) (e.g., fulvestrant), a gonadotropin-releasing hormone (GnRH) analogue, or a selective estrogen receptor modulator (SERM) (e.g., tamoxifen or raloxifene). The estrogen inhibitor preferably is tamoxifen.

Tamoxifen is a selective estrogen receptor modulator (SERM) which is indicated for the treatment of metastatic breast cancer in women and men and ductal carcinoma in situ. Tamoxifen a nonsteroidal agent that binds to estrogen receptors (ER), inducing a conformational change in the receptor, which results in a blockage or change in the expression of estrogen-dependent genes. Prolonged binding of tamoxifen to the nuclear chromatin of estrogen-dependent genes results in reduced DNA polymerase activity, impaired thymidine utilization, blockade of estradiol uptake, and decreased estrogen response. Like most SERMs, tamoxifen is antiestrogenic in breast tissue, but is estrogenic in the uterus and bone. Tamoxifen is described in detail in, for example, Jordan, V. C., Br J Pharmacol., 147 (Suppl 1): S269-76 (2006); and U.S. Pat. No. 4,536,516.

The invention includes a prime and boost protocol. In particular, the protocol includes an initial “prime” with a composition comprising a HDAC inhibitor and optionally one or more immunotherapeutic agents (e.g., cancer vaccines) followed by one or preferably multiple “boosts” with a composition containing one or more immunotherapeutic agents (e.g., cancer vaccines) and optionally a HDAC inhibitor.

When a HDAC inhibitor is administered with one or more immunotherapeutic agents (e.g., vaccines, such as cancer vaccines), the HDAC inhibitor and one or more immunotherapeutic agents (e.g., cancer vaccines) can be coadministered to the mammal. By “coadministering” is meant administering one or more immunotherapeutic agents (e.g., cancer vaccines) and the HDAC inhibitor sufficiently close in time such that the HDAC inhibitor can enhance the effect of the one or more immunotherapeutic agents (e.g., cancer vaccines). In this regard, the HDAC inhibitor can be administered first and the one or more immunotherapeutic agents (e.g., cancer vaccines) can be administered second, or vice versa. Alternatively, the HDAC inhibitor and the one or more immunotherapeutic agents (e.g., cancer vaccines) can be administered simultaneously.

The combination of the HDAC inhibitor and immunotherapy can be administered to a subject by various routes including, but not limited to, subcutaneous, intramuscular, intradermal, intraperitoneal, intravenous, and intratumoral. When multiple administrations are given, the administrations can be at one or more sites in a subject.

Administration of the combination can be “prophylactic” or “therapeutic.” When provided prophylactically, the combination is provided in advance of tumor formation to allow the host's immune system to fight against a tumor that the host is susceptible of developing. For example, hosts with hereditary cancer susceptibility are a preferred group of patients treated with such prophylactic immunization. The prophylactic administration of a HDAC inhibitor or a composition thereof (e.g., including a vaccine) prevents, ameliorates, or delays cancer. When provided therapeutically, the combination is provided at or after the diagnosis of cancer. When the host has already been diagnosed with cancer (e.g., metastatic cancer), the combination can be administered in conjunction with other therapeutic treatments such as chemotherapy or radiation.

The following formulations for oral, aerosol, parenteral (e.g., subcutaneous, intravenous, intraarterial, intramuscular, intradermal, interperitoneal, and intrathecal), rectal, and vaginal administration are merely exemplary and are in no way limiting.

Formulations suitable for oral administration can consist of (a) liquid solutions, such as an effective amount of the compound dissolved in diluents, such as water, saline, or orange juice; (b) capsules, sachets, tablets, lozenges, and troches, each containing a predetermined amount of the active ingredient, as solids or granules; (c) powders; (d) suspensions in an appropriate liquid; and (e) suitable emulsions. Liquid formulations may include diluents, such as water and alcohols, for example, ethanol, benzyl alcohol, and the polyethylene alcohols, either with or without the addition of a pharmaceutically acceptable surfactant, suspending agent, or emulsifying agent. Capsule forms can be of the ordinary hard- or soft-shelled gelatin type containing, for example, surfactants, lubricants, and inert fillers, such as lactose, sucrose, calcium phosphate, and cornstarch. Tablet forms can include one or more of lactose, sucrose, mannitol, corn starch, potato starch, alginic acid, microcrystalline cellulose, acacia, gelatin, guar gum, colloidal silicon dioxide, croscarmellose sodium, talc, magnesium stearate, calcium stearate, zinc stearate, stearic acid, and other excipients, colorants, diluents, buffering agents, disintegrating agents, moistening agents, preservatives, flavoring agents, and pharmacologically compatible carriers. Lozenge forms can comprise the active ingredient in a flavor, usually sucrose and acacia or tragacanth, as well as pastilles comprising the active ingredient in an inert base, such as gelatin and glycerin, or sucrose and acacia, emulsions, gels, and the like containing, in addition to the active ingredient, such carriers as are known in the art.

The combination of the HDAC inhibitor and immunotherapy can be made into aerosol formulations to be administered via inhalation. These aerosol formulations can be placed into pressurized acceptable propellants, such as dichlorodifluoromethane, propane, nitrogen, and the like. They also may be formulated as pharmaceuticals for non-pressured preparations, such as in a nebulizer or an atomizer.

Formulations suitable for parenteral administration include aqueous and non-aqueous, isotonic sterile injection solutions, which can contain anti-oxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. The HDAC inhibitor, immunotherapeutic agent, and/or compositions thereof can be administered in a physiologically acceptable diluent in a pharmaceutical carrier, such as a sterile liquid or mixture of liquids, including water, saline, aqueous dextrose and related sugar solutions, an alcohol, such as ethanol, isopropanol, or hexadecyl alcohol, glycols, such as propylene glycol or polyethylene glycol, glycerol ketals, such as 2,2-dimethyl-1,3-dioxolane-4-methanol, ethers, such as poly(ethyleneglycol) 400, an oil, a fatty acid, a fatty acid ester or glyceride, or an acetylated fatty acid glyceride with or without the addition of a pharmaceutically acceptable surfactant, such as a soap or a detergent, suspending agent, such as pectin, carbomers, methylcellulose, hydroxypropylmethylcellulose, or carboxymethylcellulose, or emulsifying agents and other pharmaceutical adjuvants.

Oils, which can be used in parenteral formulations include petroleum, animal, vegetable, or synthetic oils. Specific examples of oils include peanut, soybean, sesame, cottonseed, corn, olive, petrolatum, and mineral. Suitable fatty acids for use in parenteral formulations include oleic acid, stearic acid, and isostearic acid. Ethyl oleate and isopropyl myristate are examples of suitable fatty acid esters.

Suitable soaps for use in parenteral formulations include fatty alkali metal, ammonium, and triethanolamine salts, and suitable detergents include (a) cationic detergents such as, for example, dimethyl dialkyl ammonium halides, and alkyl pyridinium halides, (b) anionic detergents such as, for example, alkyl, aryl, and olefin sulfonates, alkyl, olefin, ether, and monoglyceride sulfates, and sulfosuccinates, (c) nonionic detergents such as, for example, fatty amine oxides, fatty acid alkanolamides, and polyoxyethylene-polypropylene copolymers, (d) amphoteric detergents such as, for example, alkyl-beta-aminopropionates, and 2-alkyl-imidazoline quaternary ammonium salts, and (3) mixtures thereof.

Suitable preservatives and buffers can be used in such formulations. In order to minimize or eliminate irritation at the site of injection, such compositions may contain one or more nonionic surfactants having a hydrophile-lipophile balance (HLB) of from about 12 to about 17. The quantity of surfactant in such formulations ranges from about 5% to about 15% by weight. Suitable surfactants include polyethylene sorbitan fatty acid esters, such as sorbitan monooleate and the high molecular weight adducts of ethylene oxide with a hydrophobic base, formed by the condensation of propylene oxide with propylene glycol. The parenteral formulations can be presented in unit-dose or multi-dose sealed containers, such as ampoules and vials, and can be stored in a freeze-dried (lyophilized) condition requiring only the addition of the sterile liquid carrier, for example, water, for injections, immediately prior to use. Extemporaneous injection solutions and suspensions can be prepared from sterile powders, granules, and tablets.

The HDAC inhibitor, immunotherapeutic agent, and/or compositions thereof can be administered as an injectable formulation. The requirements for effective pharmaceutical carriers for injectable compositions are well known to those of ordinary skill in the art. See Pharmaceutics and Pharmacy Practice, J. B. Lippincott Co., Philadelphia, Pa., Banker and Chalmers, eds., pages 238-250 (1982), and ASHP Handbook on Injectable Drugs, Toissel, 4th ed., pages 622-630 (1986).

Topical formulations, including those that are useful for transdermal drug release, are well known to those of skill in the art and are suitable in the context of the invention for application to skin.

The HDAC inhibitor, immunotherapeutic agent, and/or compositions thereof can be administered as a suppository by mixing with a variety of bases, such as emulsifying bases or water-soluble bases. Formulations suitable for vaginal administration may be presented as pessaries, tampons, creams, gels, pastes, foams, or spray formulas containing, in addition to the active ingredient, such carriers as are known in the art to be appropriate.

Methods for preparing administrable (e.g., parenterally administrable) HDAC inhibitors, immunotherapeutic agents, and/or compositions thereof are known or apparent to those skilled in the art and are described in more detail in, for example, Remington's Pharmaceutical Science (17th ed., Mack Publishing Company, Easton, Pa., 1985).

In addition to the aforedescribed pharmaceutical compositions, the HDAC inhibitor, immunotherapeutic agent, and/or compositions thereof can be formulated as inclusion complexes, such as cyclodextrin inclusion complexes, or liposomes. Liposomes can serve to target the HDAC inhibitor, immunotherapeutic agent, and/or compositions thereof to a particular tissue. Liposomes also can be used to increase the half-life of the the HDAC inhibitor, immunotherapeutic agent, and/or compositions thereof. Many methods are available for preparing liposomes, as described in, for example, Szoka et al., Ann. Rev. Biophys. Bioeng., 9, 467 (1980) and U.S. Pat. Nos. 4,235,871, 4,501,728, 4,837,028, and 5,019,369.

The invention further provides a kit that contains the HDAC inhibitor and immunotherapeutic agent (e.g., in one or more compositions with a pharmaceutically acceptable carrier). The kit further provides containers, injection needles, and instructions on how to use the kit.

The following examples further illustrate the invention but, of course, should not be construed as in any way limiting its scope.

### Example 1

This example provides the materials and methods for the remaining Examples.

Tumor Cell Lines

Human carcinoma cells of breast [MDA-MB-231 (ATCC® HTB-26™)], lung [NCI-H460 (ATCC® HTB-177™)], prostate [LNCaP clone FGC (ATCC® CRL-1740™)], and pancreas [AsPC-1 (ATCC® CRL-1682™)] origin were obtained from American Type Culture Collection (ATCC) and cultured in medium designated by the provider for propagation and maintenance. All cell lines were used at low passage number and proven free of Mycoplasma.

Chemicals and Drug Exposure

Vorinostat and entinostat were obtained from Selleck Chemicals. Adherent tumor cells in log-growth phase were exposed daily to vehicle (DMSO) or vorinostat at the indicated concentrations for 5 h, over 4 consecutive days. At the end of each treatment, cells were washed in fresh medium and returned to incubation at 37° C. with 5% CO₂. Alternatively, cells were continuously exposed to vehicle (DMSO) or entinostat at the indicated concentrations for 72 h.

Analysis of Cell Growth and Viability

Tumor cells were exposed to DMSO or vorinostat as described above. Cells were harvested daily and viable cells were counted by trypan blue exclusion using a Cellometer Auto T4 automated cell counter (Nexcelom Bioscience). Cellular viability was confirmed by flow cytometry using Live/Dead exclusion, according to manufacturer's instructions (Invitrogen).

HDAC Activity Assay

Changes in the nuclear enzyme activity of HDAC isoform is 1-11 following vorinostat treatment of MDA-MB-231 and LNCaP cells were determined using the colorimetric EpiQuik HDAC Activity/Inhibition Assay Kit (Epigentek). Briefly, 10 μg of extracted nuclear HDAC proteins were incubated with acetylated HDAC substrate for 90 min at 37° C. HDAC deacetylated products were detected following sequential incubation with capture and detection antibodies, according to the manufacturer's specifications.

CD8⁺ Cytotoxic T-Cell (CTL) Lines

Carcinoembryonic antigen (CEA)-specific CTLs recognize the CEA peptide epitope YLSGANLNL (CAP-1) (SEQ ID NO: 1) (Tsang et al., J. Natl. Cancer Inst., 87(13): 982-990 (1995)). Prostate-specific antigen (PSA)-specific CTLs recognize the PSA peptide epitope VLSNDVCAQV (SEQ ID NO: 2) (Correale et al., J. Natl. Cancer Inst., 89(4): 293-300 (1997)). The mucin-1 (MUC-1)-specific CD8⁺ CTL line, designated MUC-1 CTL, recognizes the MUC-1 peptide epitope ALWGQDVTSV (SEQ ID NO: 3) (Tsang et al., Clin. Cancer Res., 10(6): 2139-2149 (2004)). Brachyury-specific CTLs recognize the brachyury peptide epitope WLLPGTSTL (T-p2) (SEQ ID NO: 4) (Tucker et al., Cancer Immunol. Immunother., 63(12): 1307-1317 (2014)). All T-cell lines were HLA-A2-restricted.

Cytotoxicity Assays

Carcinoma cells exposed to vorinostat, entinostat, or vehicle (DMSO) were labeled with ¹¹¹In prior to being used as targets for direct lysis by effector CTLs at an effector-to-target ratio of 30:1 in a standard overnight cytotoxicity ¹¹¹In-release assay (Gameiro et al., Oncoimmunology, 3: e28643 (2014)).

Gene Silencing and Western Blots

Silencer® siRNA and negative control siRNA were used to silence HDAC1, ERN1, and PERK in MDA-MB-231 carcinoma cells, according to the manufacturer's instructions (Life Technologies). Cells were exposed to siRNA 24 h prior to treatment with vorinostat or DMSO for 4 consecutive days, as described above. At the end of treatment, cells were harvested and used as CTL targets. The expression level of targeted proteins was examined by Western blotting of cell lysates prepared in RIPA buffer containing 1 mM PMSF (Cell Signaling Technology). Proteins (20-40 μg) were separated using 4%-12% MOPS SDS-PAGE (Life Technologies) then transferred to nitrocellulose membranes. Primary antibodies specific for HDAC1, ERN1, PERK, and GAPDH were acquired from Cell Signaling Technology. Blots were incubated with anti-rabbit IRDye secondary antibodies (LI-COR Biotechnology). Detection and quantification of band intensity were performed with the Odyssey Infrared Imaging System (LI-COR Biotechnology). Protein levels were normalized to the loading control GAPDH.

Luciferase ER Stress Reporter Assays

Human prostate carcinoma LNCaP cells were stably transduced with replicant-incompetent lentiviral particles expressing an inducible reporter construct encoding the firefly luciferase gene under the control of a basal promoter element (TATA box) joined to tandem repeats of the endoplasmic reticulum (ER) stress transcriptional response element (ERSE) (Qiagen). As an internal control, cells were co-transduced with lentiviral particles expressing a constitutive Renilla luciferase expression cassette under the control of the CMV promoter (Qiagen). Transduced cells were selected in media containing 1 μg/ml puromycin (Life Technologies) and single-cell clones were selected for study. Luciferase activity was quantified using the Dual-Luciferase Reporter Assay (Promega).

Flow Cytometry Analysis

Cell-surface and intracytoplasmic staining was performed as previously described (Ogino et al., J. Immunol. Methods, 278(1-2): 33-44 (2003)). Surface staining of tumor cells was performed using the primary labeled monoclonal antibodies HLA-A2-FITC, ICAM-1 (CD54)-PE, CEA (CD66)-FITC, MUC-1 (CD227)-FITC, and the appropriate isotype-matched controls (BD Biosciences). For intracellular analysis of antigen processing machinery (APM) components, mouse IgG1 (MK2-23) isotype control, LMP2 (SY-1)-, LMP7 (HB2)-, TAP-1 (NOB1)-, calnexin (TO-5)-, β2-microglobulin (L368), and tapasin (TO-3)-specific monoclonal antibodies were developed and characterized as described (Bandoh et al., Tissue Antigens, 66(3): 185-194 (2005); Ogino et al., Tissue Antigens, 62(5): 385-393 (2003); and Wang et al., J. Immunol. Methods, 299(1-2): 139-151 (2005)). Cellular fluorescence of 3×10⁴ cells was examined on a FACSCalibur flow cytometer using CellQuest software (BD Biosciences). Proteins were scored as markedly upregulated if confirmed statistically and if detection levels and/or mean fluorescence intensity (MFI) increased ≥25% following treatment and were not observed in control cells vs. isotype controls.

Statistical Analysis

The effect of repetitive drug exposure over time on cellular proliferation was examined by 2-way ANOVA. Significant differences between multiple treatment groups were determined by 1-way ANOVA with Tukey's comparison, both based on a confidence interval of 95% using Prism 6.0f software (GraphPad Software Inc.). Alternatively, statistical differences between 2 treatments were analyzed by unpaired Student's t test with a 2-tailed distribution, unless reported otherwise, and reported as P values. Significant differences in the distribution of flow cytometry analysis data were determined by the Kolmogorov-Smirnov test using CellQuest software (BD Biosciences).

### Example 2

This example demonstrates that vorinostat decreases pan-HDAC activity and proliferation of human carcinoma cells in an exposure-dependent manner.

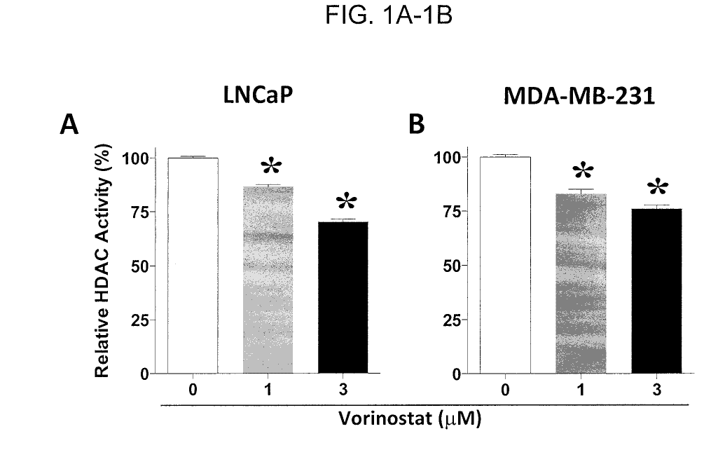

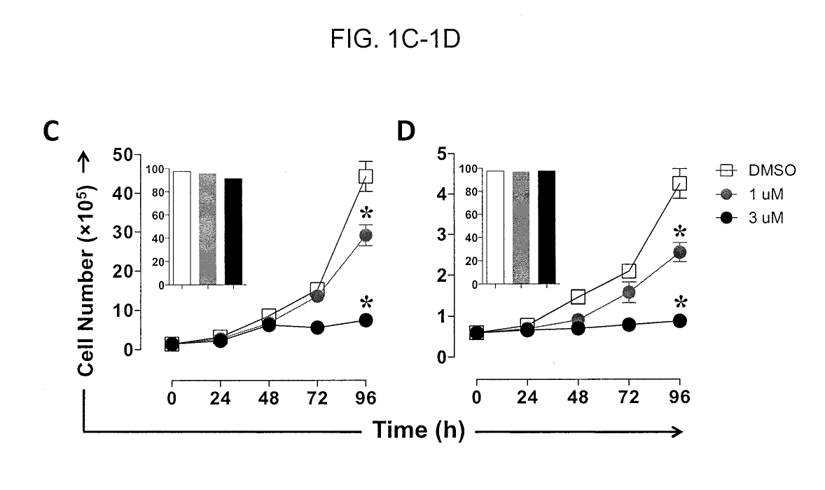

Supra clinical exposure of tumor cells to HDAC inhibitors, including vorinostat, has been shown to inhibit Class I and Class II histone deacetylases as well as exert antiproliferative effects (Butler et al., Cancer Res., 60(18): 5165-5170 (2000); and Richon et al., Proc. Natl. Acad. Sci. USA, 97(18): 10014-10019 (2000)). Therefore, the in vitro effect of clinically relevant exposure of human prostate (LNCaP) and breast (MDA-MB-231) carcinoma cells to vorinostat on the activity of HDAC enzymes (isoforms 1-11), cellular proliferation, and viability was examined. Tumor cells were exposed daily for 5 h to 1 μM or 3 μM vorinostat, or vehicle (DMSO) over 4 consecutive days, mimicking the range of vorinostat exposure (Cmax, AUC) attained in cancer patients after oral once daily intake of 400 mg (Iwamoto et al., Cancer Chemother. Pharmacol., 72(3): 493-508 (2013)). Exposure to vorinostat significantly decreased HDAC activity in a dose-dependent manner in both prostate (FIG. 1A, P=0.0006) and breast (FIG. 1B, P=0.0046) carcinoma cells. In addition, significantly decreased cellular proliferation was also observed in a dose-dependent manner after exposure to vorinostat in both prostate (FIG. 1C, P<0.0001) and breast (FIG. 1D, P<0.0001) carcinoma cells relative to vehicle controls, with no significant effect observed on cellular viability (FIG. 1C-D insets). These data indicate that clinically relevant exposure of prostate and breast carcinomas to vorinostat induces target inhibition and promotes slower tumor growth. Vorinostat concentration of 3 μM was used for all subsequent experiments.

### Example 3

This example demonstrates that carcinoma cells exposed to vorinostat are significantly more sensitive to CTL-mediated killing.

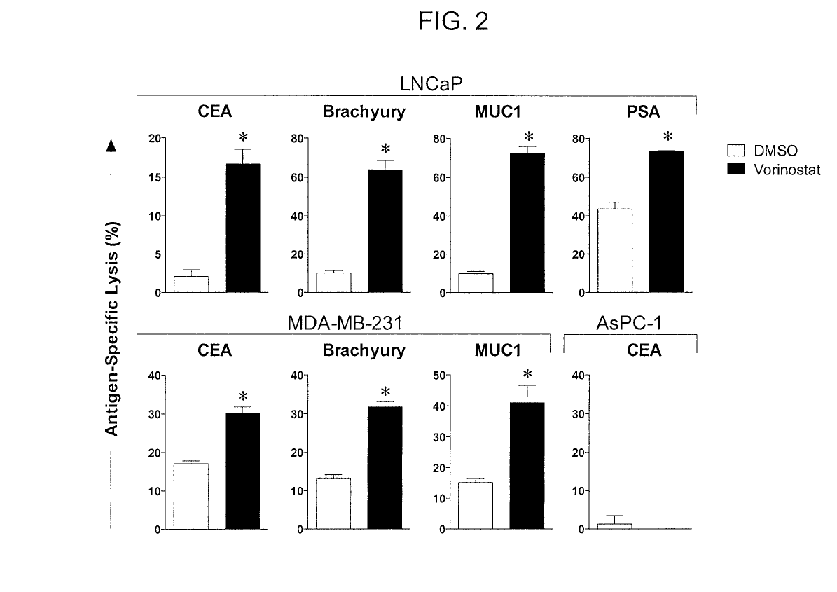

The effect of clinically relevant vorinostat exposure on prostate and breast carcinoma cells' sensitivity to antigen-specific CTL-mediated lysis was examined. LNCaP and MDA-MB-231 were exposed to vorinostat or to vehicle as before, prior to being used as targets for antigen-specific CTL lysis, using CEA-, brachyury-, MUC-1-, or PSA-specific CD8⁺ T cells as effector cells. As shown in FIG. 2, prostate carcinoma cells were significantly more sensitive to CTL-mediated lysis targeting CEA (P=0.002), brachyury (P=0.0004), MUC-1 (P<0.0001), or PSA (P=0.0011). Similar results were observed with MDA-MB-231 breast carcinoma cells treated with vorinostat. The absence of significant lysis of HLA-A2 negative AsPC-1 pancreatic carcinoma cells exposed to vehicle or vorinostat confirmed that all effector T cells were HLA-A2 restricted.

Similar results were observed with additional cell lines representative of distinct tumor types, including breast (MCF-7, ER+) and colon (SW620 and SW480) carcinomas.

These data show that treatment of solid carcinomas with clinically relevant vorinostat exposures enhances antigen-specific CTL-mediated killing against a variety of tumor-associated antigens (TAAs) and across different tumor types, indicating a broad increase in tumor recognition by T cells.

### Example 4

This example demonstrates that vorinostat induces immunogenic modulation in carcinoma cells, including increased APM component expression.

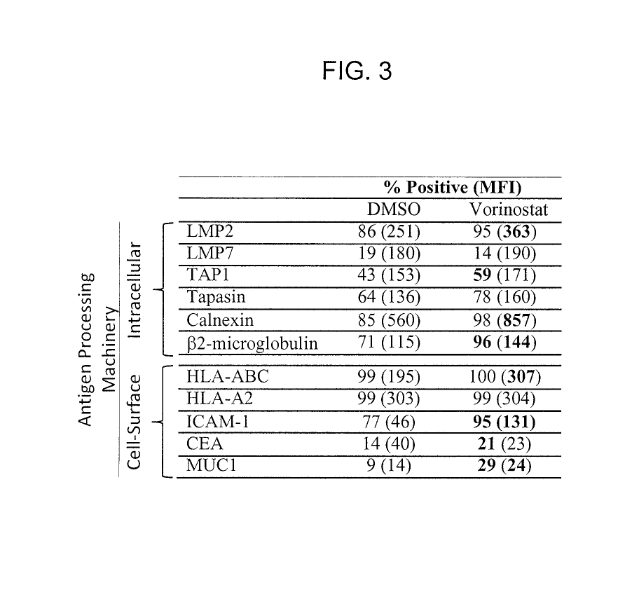

CTL killing of tumor targets requires T-cell recognition of specific major histocompatibility complex (MHC) Class I/CD8⁺-restricted epitope complexes on the surface of tumor cells, an event determined by the cooperative interactions of multiple APM components. This suggests that the increased CTL-mediated lysis of tumor cells observed upon exposure to vorinostat may be a consequence of APM component upregulation. To test this hypothesis, MDA-MB-231 carcinoma cells were exposed to vorinostat or to vehicle as before. At the end of treatment, cells were examined by flow cytometry for intracellular expression of 6 APM components (FIG. 3).

Exposure to vorinostat significantly increased the expression of 5 APM components by ≥25%, namely the immune proteosome subunits LMP2 and LMP7, the peptide transporter TAP1, the chaperone calnexin, and the HLA class I heavy chain-associated β2-microglobulin. Tapasin expression was also increased (22%) albeit to a lesser degree. Increased expression of HLA class I antigens and ICAM-1 was observed, as well as the TAAs CEA and MUC-1 on the surface of tumor cells upon exposure to vorinostat.

These data indicate that HDAC inhibition upregulates multiple APM components; this change is likely to enhance the synthesis and expression of HLA class I antigen-TAA derived peptide complexes, resulting in increased T-cell recognition and lysis of tumor targets exposed to vorinostat. In other words, clinically translatable exposure of carcinoma cells to HDAC inhibitors (e.g., vorinostat) reprograms multiple elements of the APM machinery, thereby augmenting tumor recognition and lysis by cytotoxic T cells.

### Example 5

This example demonstrates that vorinostat-induced immunogenic modulation of MDA-MB-231 carcinoma cells is mediated by HDAC1.

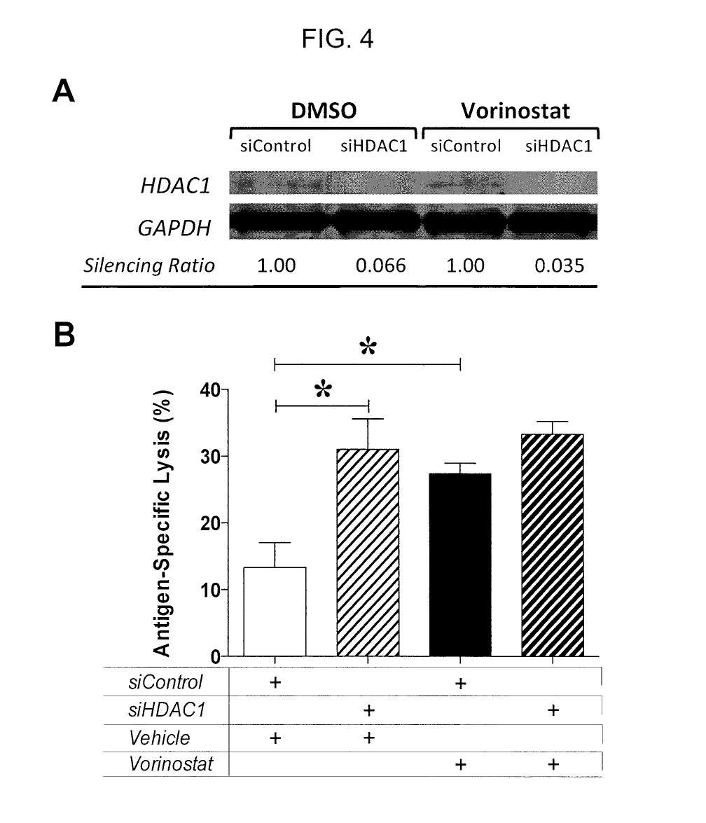

Class I HDAC1-3 are major targets of vorinostat, and have been shown to be co-repressors of gene transcription, including genes involved in tumor immune recognition (West et al., J. Clin. Invest., 124(1): 30-39 (2014); Yang et al., Epigenetics, 7(4): 390-399 (2012); and Nebbioso et al., Nat. Med., 11(1): 77-84 (2005)). This suggests that this class of HDACs mediates vorinostat-induced immunogenic modulation of tumor cells, thus rendering them more sensitive to CTL-mediated killing. To test this hypothesis, MDA-MB-231 cells were exposed to silencing RNA (siRNA) targeting HDAC1 or control siRNA for 24 h prior to exposure to vehicle or vorinostat as before. As shown in FIG. 4A, HDAC1 expression in tumor targets treated with siRNA targeting HDAC1 was significantly decreased at the end of treatment compared with targets exposed to control siRNA. At the end of treatment, tumor cells were used as targets for brachyury-specific T-cell-mediated lysis. As shown in FIG. 4B, vorinostat exposure significantly augmented CTL sensitivity of MDA-MB-231 target cells exposed to control siRNA, a 2-fold increase relative to vehicle controls (P=0.0024). Strikingly, the augmented CTL lysis attained in silencing control targets after exposure to vorinostat also was observed upon silencing of HDAC1 in the absence of vorinostat exposure. Moreover, treatment of HDAC1-silenced MDA-MB-231 tumor cells with vorinostat did not further increase CTL lysis relative to vehicle control. Altogether, this data suggest that vorinostat-induced immunogenic modulation of MDA-MB-231 breast carcinoma cells is mediated by HDAC1.

The immunogenic modulation promoted by HDAC inhibitors is a consequence of direct target inhibition as silencing HDAC1 in tumor targets increases their sensitivity to CTL killing to the same extent as pharmacological inhibition with vorinostat with no additive effect of vorinostat observed in targets with silenced HDAC1 (see FIG. 4).

### Example 6

This example demonstrates that HDAC inhibition activates the ER stress responsive element in an exposure-dependent manner.

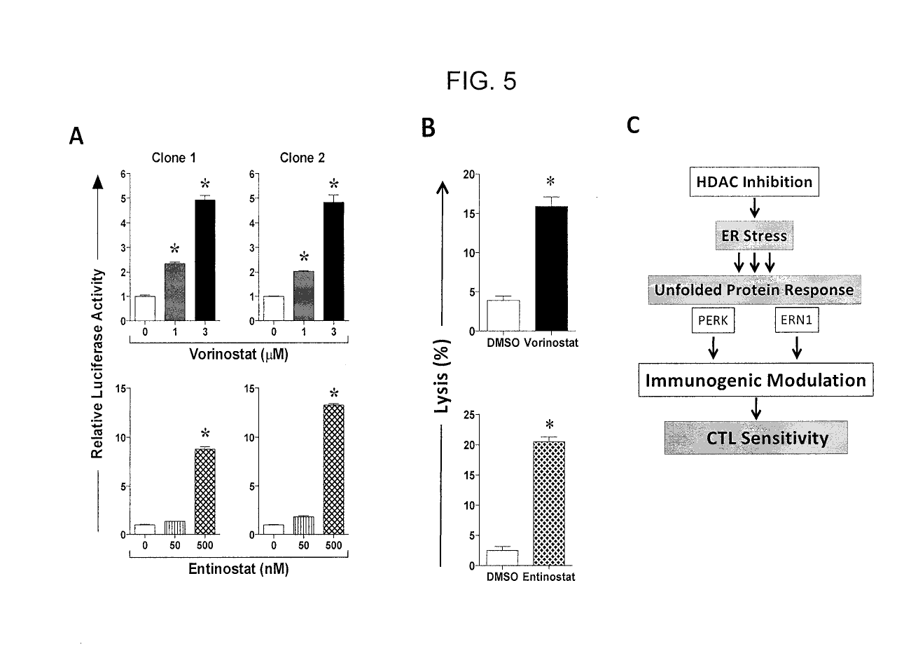

Immunogenic modulation and augmented immune recognition of human carcinoma cells by cognate cytotoxic T cells encompasses a tumor adaptive response to endoplasmic reticulum stress through the UPR (Gameiro et al., Oncotarget, 5(2): 403-416 (2014)). HDAC1, a Class I HDAC and main vorinostat target, has been shown to control the transcription of Grp78, an ER stress responsive genes by directly binding to the ER stress response element (ERSE) (Baumeister et al., Mol. Cell. Biol., 25(11): 4529-4540 (2005)). Vorinostat may therefore activate the ER stress response through HDAC1 inhibition. To test this hypothesis, two single-cell clones of LNCaP cells stably transduced with an ERSE reporter driving firefly luciferase expression were exposed to vorinostat or vehicle as before. As shown in FIG. 5A, vorinostat activated ERSE in a dose-dependent manner. To further examine the induction of ER stress through Class I HDAC inhibition, ERSE reporter clones were treated with clinically relevant exposures of entinostat, a selective Class I HDAC inhibitor (West et al., J. Clin. Invest., 124(1): 30-39 (2014)). Similarly to vorinostat, tumor exposure to entinostat activated ERSE in an exposure-dependent manner (FIG. 5A), resulting in increased sensitivity to CTL-mediated killing similar to that with vorinostat (FIG. 5B).

Altogether, this data indicates that HDAC inhibition with agents targeting Class I HDAC enzymes induces ER stress, which ultimately results in immunogenic modulation and increased tumor sensitivity to CTL-mediated lysis (FIG. 5C).

### Example 7

This example demonstrates that the unfolded protein response mediates vorinostat-induced immunogenic modulation.

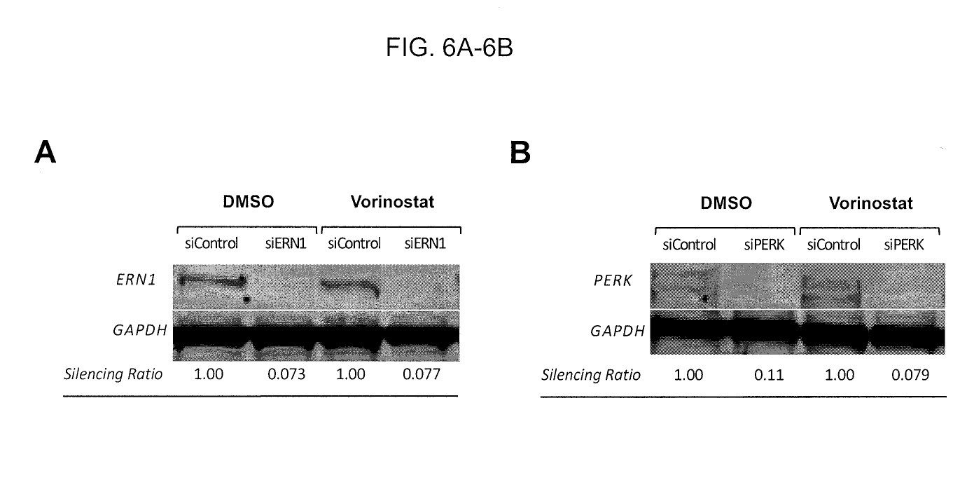

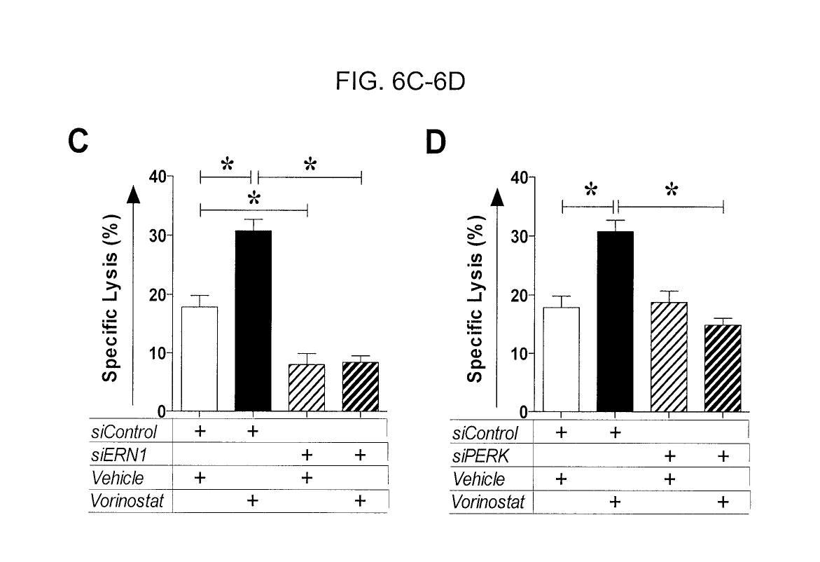

ER stress activates the UPR, an adaptive reaction attempting to restore ER homeostasis through a cascade of cellular events (Hetz et al., Nat. Cell. Biol., 17(7): 829-838 (2015)). To examine the functional consequence of ER stress induced by HDAC inhibition and the possible involvement of the UPR, MDA-MB-231 cells were exposed to siRNA control or targeting two independent ER stress/UPR sensors, ERN1 or PERK, for 24 h prior to being exposed to vehicle or vorinostat as before. At the end of treatment, gene silencing was confirmed (FIG. 6A-B) and tumor cells were used as targets for CEA-specific CTL lysis (FIG. 6C-D). As shown in FIG. 6C, exposing MDA-MB-231 cells to control siRNA led to significantly increased target lysis by cytotoxic T cells following vorinostat treatment (P<0.0001). However, vorinostat did not increase CTL lysis of tumor cells when ERN1 (FIG. 6C) or PERK (FIG. 6D) were silenced.

Collectively, these data suggest that the increased sensitivity of human carcinoma cells to CTL-mediated lysis as a result of HDAC inhibition stems from a cellular survival response to ER stress mediated through the UPR.

### Example 8

This example demonstrates HDAC inhibition increases sensitivity to NK mediated killing and enhances ADCC.

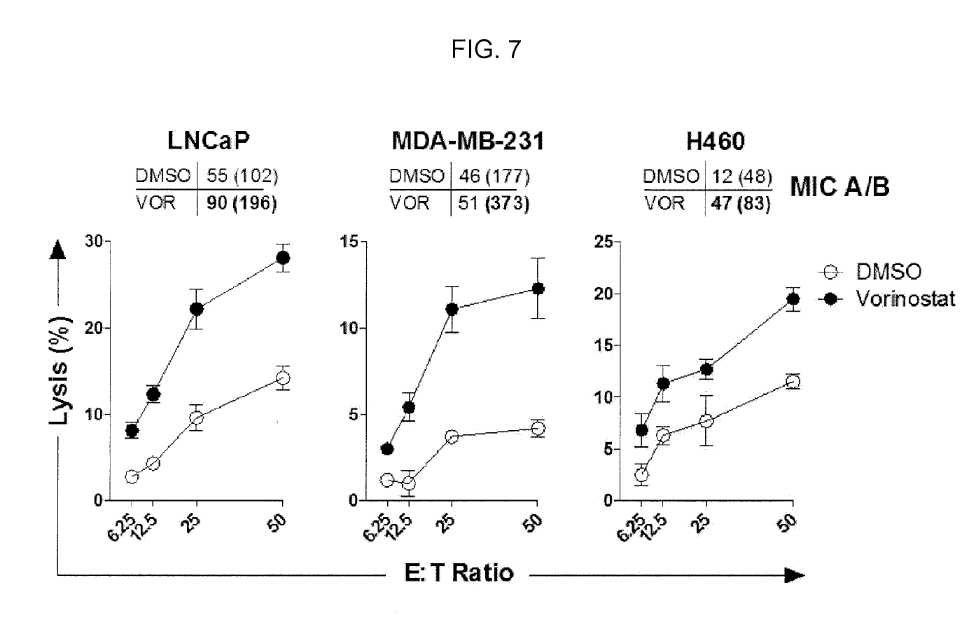

Human prostate (LNCaP), breast (MDA-MB-231), and lung (H460) carcinoma cells were exposed to vorinostat or vehicle (DMSO) and the percent lysis was determined. At the end of treatment, the cells were used as targets in a lysis assay wherein NK cells isolated from human PBMCs were used as effectors (varying E:T ratios as indicated in FIG. 7). The effect of vorinostat on sensitivity of human carcinoma cells to NK killing is demonstrated in FIG. 7.

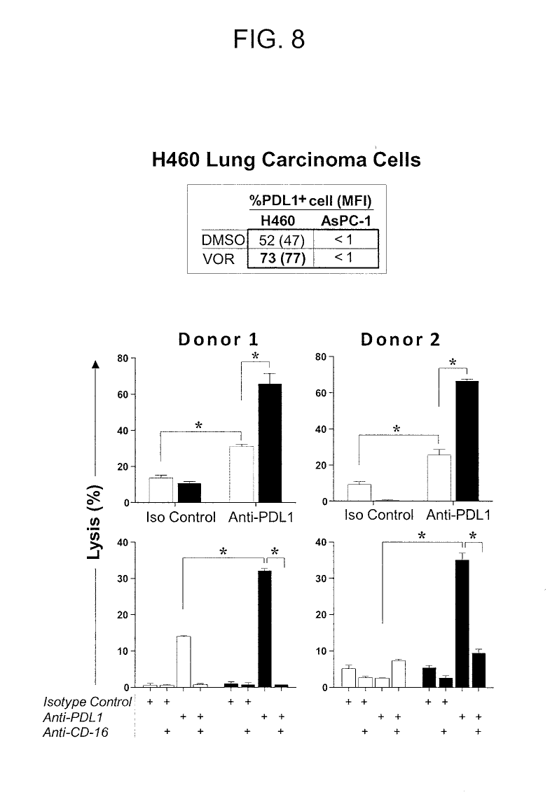

Human lung (H460) carcinoma cells were exposed to vorinostat and isotype control, anti-CD16, or anti-PD-L1 (avelumab). As shown in FIG. 8, vorinostat increases avelumab-mediated ADCC.

Similar results were observed with entinostat.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A method of reducing cancer cell growth, which method comprises treating cancer cells with a combination of a histone deacetylase (HDAC) inhibitor and immunotherapy, whereupon growth of the cancer cells is reduced.

2. A method of increasing sensitivity of cancer cells to cytotoxic T-cell (CTL) mediated killing, which method comprises treating cancer cells with a combination of a histone deacetylase (HDAC) inhibitor and immunotherapy, whereupon the sensitivity of the cancer cells to CTL mediated killing is increased.

3. A method of increasing sensitivity of cancer cells to natural killer (NK) cell mediated killing, which method comprises treating cancer cells with a combination of a histone deacetylase (HDAC) inhibitor and immunotherapy, whereupon the sensitivity of the cancer cells to NK mediated killing is increased.

4. The method of claim 1, wherein the cancer cells are from a solid tumor.

5. The method of claim 1, wherein the cancer cells are prostate cancer cells, breast cancer cells, lung cancer cells, or colon cancer cells.

6. The method of claim 1, wherein the HDAC inhibitor is a class I HDAC inhibitor.

7. The method of claim 6, wherein the HDAC inhibitor is vorinostat.

8. The method of claim 6, wherein the HDAC inhibitor is entinostat.

9. The method of claim 1, wherein the immunotherapy is a checkpoint inhibitor, vaccine, a monoclonal antibody, a cell-based immunotherapy, or a radiopharmaceutical.

10. The method of claim 9, wherein the immunotherapy is a vaccine and the vaccine is a virus-based vaccine.

11. The method of claim 10, wherein the virus-based vaccine is a poxviral-based vaccine.

12. The method of claim 11, wherein the immunotherapy is the PSA/TRICOM vaccine (PROSTVAC™).

13. The method of claim 10, wherein the virus-based vaccine is an adenoviral-based vaccine.

14. The method of claim 10, wherein the immunotherapy is a MUC-1/CEA vaccine.

15. The method of claim 10, wherein the immunotherapy is a Brachyury vaccine.

16. The method of claim 9, wherein the immunotherapy is Sipuleucel-T (PROVENGE™), ipilumimab, nivolumab, radium-223 (XOFIGO™), a yeast-MUC-1 immunotherapeutic, or trastuzumab (HERCEPTIN™).

17. The method of claim 1, further comprising treating the cancer cells with one or more additional therapeutic agents.

18. The method of claim 17, wherein the one or more additional therapeutic agents are enzalutamide or abiraterone.

19. The method of claim 1, wherein the cancer cells are in vivo.

20. The method of claim 19, wherein the cancer cells are in a human.

21. The method of claim 1, wherein the cancer cells are in vitro.
